(19) United States
(12) Patent Application Publication
(10) Pub. No.: US 2007/0015974 A1
(43) Pub. Date: Jan. 18, 2007

(54) Methods and systems for monitoring and enhancing patient compliance with a health treatment program

(75) Inventors: Rose Higgins, Washington Crossing, PA (US); Brett Allen Olive, San Diego, CA (US); Timothy Bailey, Escondido, CA (US)

Correspondence Address: BEYER WEAVER & THOMAS, LLP, P.O. BOX 70250, OAKLAND, CA, 94612-0250, US

(73) Assignee: iMetrikus, Inc., Carlsbad, CA (US)

(21) Appl. No.: 11/432,655

(22) Filed: May 11, 2006

Related U.S. Application Data
(60) Provisional application No. 60/679,891, filed on May 11, 2005.

(51) Int. Cl. A61B 5/00

(52) U.S. Cl. 600/300

(57) ABSTRACT

Methods and systems for enabling and supporting patient compliance with a health treatment program or with health-related instructions from a caregiver are described. An interactive feedback loop is implemented that enhances and improves on present methods of monitoring and supporting patient compliance with treatment programs, particularly for patients with chronic conditions. A patient enters various types of data (e.g., biometric readings, diary entries, responses to surveys and health assessments, etc.) to a health data management system that includes a patient compliance monitoring system. The patient is encouraged to enter as much data as is practical and as frequently as possible. The feedback loop of the present invention provides compliance messages to the patient in a timely manner that are responsive to the data entered by the patient. The messages are customized, supportive, and timely. The system can also implement a reward scheme in which patients who go beyond their prescribed treatment program or consistently stay within a compliance range obtain direct financial benefits or rewards. The system also allows patients to sends feedback on the messages he or she receives from the system. This feedback can be used to measure the effectiveness of a treatment program or of the monitoring system itself. Healthcare professionals play an active role in tailoring and “signing off” the compliance-related messages that the patient receives.

### CROSS-REFERENCE TO RELATED APPLICATIONS

This application claims priority of U.S. Provisional Application 60/679,891, filed May 11, 2005, entitled “Patient Compliance Monitoring System” which is incorporated by reference.

### FIELD OF THE INVENTION

The present invention relates generally to methods and systems for improving patient compliance in the healthcare field. More specifically, the present invention relates to methods of monitoring, encouraging, and motivating healthcare consumers to comply with treatment programs, medication, health-related regimens, and so on, by providing balanced, customized and timely feedback to the consumer.

### BACKGROUND OF THE INVENTION

Healthcare costs are increasing at an alarming rate and no entity in the healthcare industry is immune. Patients, doctors, doctor groups, hospitals, government agencies and insurance companies are all experiencing higher costs.

One approach that has been taken to reducing costs is improving patient compliance to treatment programs or, more generally, to health and medical instructions and advice from a healthcare provider. The underlying strategy of this approach is to reduce the likelihood of patients returning to the healthcare care system, especially to the costly aspects of the system, such as hospital stays, surgery, and so on. In order to prevent this patient “bounce back” into the healthcare system, specifically resulting from a patient not complying with health instructions or health treatment program, methods and frameworks are used to ensure that a patient follows a doctor's instructions.

Patients with chronic illnesses (e.g., patients with asthma, high-blood pressure, diabetes, heart disease, HIV, and so on) require more healthcare if they do not follow a prescribed regimen, which may involve biometric readings on regular basis, taking medication, following a strict exercise and diet regimen, performing physical therapy, and so on. Lack of adherence is a particular problem for such patients or with patients who require long-term continuous care, and less so for patients, with acute or sub-acute conditions that can be treated at a single episode of care.

One method to ensure that a patient adheres to best care practices and is compliant is to simply call the patient to follow up and see whether they are complying with a treatment program. If the patient is not, he or she is given verbal instructions (often order to be compliant) over the phone, for example, four or five times a year. Follow-up phone calls are sometimes performed by disease management organizations, often operated or set up by insurance companies, which have a strong financial incentive to keep patients who require aggressive management such as those with chronic conditions from returning to hospitals, requiring further surgery, expensive testing, re-filling prescription medications, and so on. Companies may have a registry of patients who are high financial risk and potentially expensive (e.g, for whom a return hospital visit may cost $500,000) that is derived from computer-driven models using data from claims, electronic medical records, and the like. These call center type operations focus on the high-risk patients with chronic illnesses who would cost the insurance company the most money if they were to return to the healthcare system. They typically comprise a small percentage of the healthcare consumer population. Patients who can be characterized as borderline, who are also not likely to fully comply with a treatment program, yet are not seen as a “bounce back” risk, are sent informational packets in the mail or via e-mail with instructions on how to be compliant with a treatment program for their specific condition. This technique is even less interactive and effective than receiving instructions or reminders over the phone.

Another method of maintaining compliance by a patient with a chronic illness is enrolling the patient in a support group for patients with that condition. Although this is a more interactive means for encouraging compliance, it requires time and discipline from the patient and while psychologically encouraging, support groups may not be an optimal way of making patients comply with a treatment program.

Other attempts by health organizations, including governmental agencies, to reach the general healthcare consumer population about the virtues of staying healthy by following diet and exercise guidelines, taking preventative medications, and so on, have more or less failed and have not proved effective in encouraging patients with chronic conditions to follow a treatment program. For example, the FDA's Food Pyramid is not likely to inspire a young patient with obesity health issues to decrease his or her daily caloric intake. Self-management certification and educational programs have also not proven effective.

Although there are some indirect financial rewards, such as deductions and benefits from insurance companies and employers, none of the present methods of encouraging patient compliance directly involve rewarding or providing some type of financial benefit to the patient for being compliant or for going beyond a compliance program and extra steps to prevent a bounce back or relapse. The greatest benefit of such compliance is of course to the patient, who's health is of paramount importance. However, there is also a financial benefit to the patient and an even greater financial benefit to entities in the healthcare industry, such as government agencies, employers, and insurance companies. However, present patient compliance methods, such as disease management organizations, do not include a framework in which entities that reap the greatest financial profit from patient compliance are the ones who reward such behavior.

Therefore, it would be desirable to have a method of improving patient compliance that involves sending customized, balanced, and timely feedback to the patient based on .a rich set of data and statistics on the patient and which allows a patient to provide a response to such feedback whereby the patient plays an integral role in the compliance process. It would also be desirable to use a patient compliance system to measure the effectiveness of compliance messages, treatment programs, and the like.

### SUMMARY OF THE INVENTION

Methods and systems for improving compliance by a patient to a healthcare treatment program and, more generally, to instructions provided by a caregiver are described. In a preferred embodiment of the present invention, the patient compliance method is used by patients with chronic conditions who require continuous, long-term care and are at risk of requiring further, often expensive, healthcare services, such as surgery and hospital stays, if not compliant with a treatment program.

In one aspect of the invention, a patient's adherence to a compliance program is measured by first having health data transmitted to a central repository or knowledge database. The health data can be of various types ranging from biometric readings direct from a home monitoring device to a short narrative on the day's physical activities, diet, unusual events, and so on. Based on these data, a healthcare provider, most often a doctor or other caregiver, and a healthcare data management system (which includes the knowledge database) make a determination as to whether the patient is being compliant. Based on this determination, a message is sent to the patient. A message may be sent if the patient is compliant or if the patient is not compliant. In a preferred embodiment of the present invention, messages sent to the patient are balanced and customized for the patient.

### BRIEF DESCRIPTION OF THE DRAWINGS

The invention will be better understood by reference to the following description taken in conjunction with the accompanying drawings in which:

FIG. 1 is a network diagram showing the basic components in the patient compliance system in accordance with one embodiment of the present invention;

FIG. 2 is a block diagram of a personal health record in accordance with one embodiment of the present invention;

FIG. 3 is a block diagram showing software logic components for a patient compliance system in accordance with one embodiment of the present invention; and

FIG. 4 is a diagram of a patient compliance monitoring feedback loop of the present invention.

### DETAILED DESCRIPTION

Reference will now be made in detail to a preferred embodiment of the invention. An example of the preferred embodiment is illustrated in the accompanying drawings. While the invention will be described in conjunction with a preferred embodiment, it will be understood that it is not intended to limit the invention to one preferred embodiment. To the contrary, it is intended to cover alternatives, modifications, and equivalents as may be included within the spirit and scope of the invention as defined by the appended claims.

Methods and systems for encouraging patients to follow healthcare treatment programs by providing customized feedback while at the same time measuring the effectiveness of the treatments are described. Methods of the present invention are interactive wherein the patient also provides feedback rather than simply being an end-point where the process stops. The process of the present invention resembles a feedback loop in which the patient is viewed not only as a receiver of instructions but as an integral source of data into the system.

One of the strategies of the patient compliance methodology of the present invention is to increase the patient's motivations to be compliant, that is, improve ways to make a patient want to be compliant with a treatment program rather than forcing or instructing him to adhere to best care practices and compliance. Generally, present methods of patient compliance involve forcing or nagging a patient to be compliant. The present invention takes a different approach.

Although the primary source of motivation of a patient to comply with a treatment program is the patient's own health and well being, other sources of motivation are sometimes needed. The methodologies of the present invention utilize positive, balanced feedback to the patient and the potential for financial reward for adhering to a treatment program.

An underlying principle of the methods and systems of the present invention, and one that has been gaining traction in the healthcare industry over many years, is that the locus of care, especially for chronic conditions should be at the home and workplace. Although in-person examinations by doctors and visits to clinics and hospitals will never be supplanted entirely by health work at the home or workplace, it is becoming increasingly important to make home healthcare more effective for many reasons, paramount of which is improving the condition of the patient.

Although the patient compliance monitoring system of the present invention is described in the context of patients with a chronic illness or ailment, following a specific treatment program, it can be used with healthcare consumers who want to follow a healthy lifestyle, practice a preventive healthcare program, or, in a specific example, are training for a particular sporting event or physical activity.

In a preferred embodiment, the patient compliance system is implemented using a feedback loop that does not require in-person visits with a caregiver. One or more devices are used to measure a patient's biometric readings at home or other remote locations such as the patient's workplace. These biometric readings are transmitted to a knowledge database where they are stored in a patient's personal health record. Such a device is described in pending application, application Ser. No. 09/977,472, titled “Method and Apparatus for Communicating Data Between a Medical Device and a Central Data Repository”, which is hereby incorporated by reference for all purposes. A personal health record, knowledge database, and healthcare data management system are described in pending application, application Ser. No. 10/417,794, titled “Methods and System for Communication and Collaboration Between a Patient and Healthcare Professional”, which is incorporated by reference for all purposes. An example of a device is MetrikLink®, used in conjunction with home monitoring devices, and an example of an online healthcare data management system is MediCompassConnect, both implemented by and available from iMetrikus, Inc. of Carlsbad, Calif.

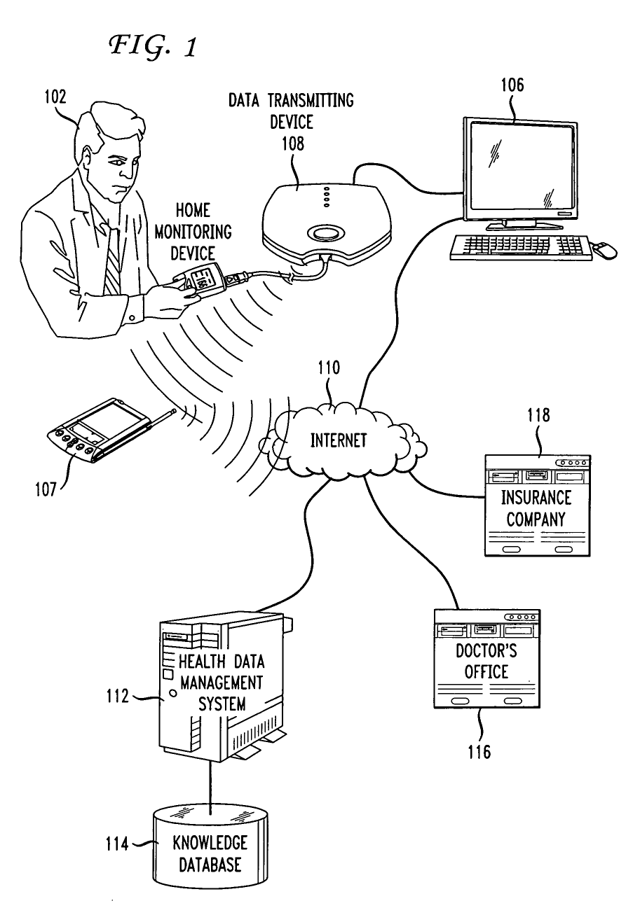

FIG. 1 is a network diagram showing the basic components in the patient compliance system in accordance with one embodiment of the present invention. A patient 102 uses a home monitoring device 104, a computer 106 and/or other IP-enabled mobile device 107, such as a wireless mobile device, and a biometric data transmitting device 108 (e.g., MetrikLink) to collect biometric data and enter, using computer 106 or a telephone connection (not shown), other types of data and information. These data and information are transmitted over a network, such as Internet 110, to healthcare data management system 112 that has a knowledge database 114 that stores personal health records (not shown). Healthcare providers, such as doctors 116 or other caregivers, and other healthcare entities such as insurance companies 118 can access system 112 and database 114 using computers or other IP-enabled devices. They can also send data, such as messages, directly to patient 102 via computer 106 or to a mobile communication device 107 such as a cell phone, pager, and so on. Data and information on patient activity, routines, and so on can also be entered by other healthcare providers, such as nurses, health coaches, trainers, pharmacists, and physical therapists.

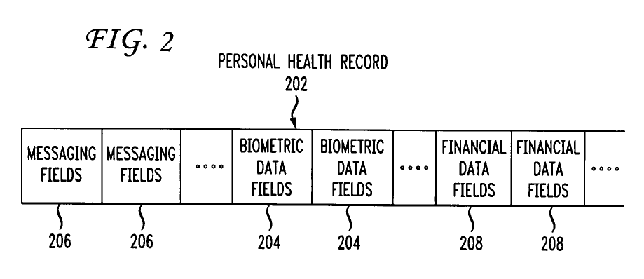

As mentioned above, knowledge database 114 contains a personal health record for each patient in the compliance system of the present invention. FIG. 2 is a block diagram of a personal health record in accordance with one embodiment of the present invention. Record 202 contains all types of data, ranging from data that can be highly validated and calibrated, to pure narrative information entered by a patient similar to an entry in a health journal. Data included in a personal health record include: biometric readings stored in biometric data fields 204, narrative information (e.g., text entered by a patient on their daily activities) in messaging fields 206, and data relating to insurance claims and various other types of health-related financial data. The total number of fields in a personal health record 202 can vary widely and can easily reach into the hundreds. There may be other categories of fields. The ones shown in FIG. 2 are illustrative. Data stored in biometric data fields 204 and information stored in messaging fields 206 can be seen as examples of content provided by a patient that lies at two opposite ends of a spectrum of data/content types that can be stored in or associated with personal health record 202 accessible in knowledge database 114. More generally, data at one end of the data/content spectrum can be fully validated, measured, reproducible, calibrated and, in some cases, regulated. These data are easily processed and have an automated aspect to them. For example, they are well suited for basing patient compliance rewards. At the other end of the spectrum are data or information that cannot be easily validated or processed and are not the result of an automated process. In many cases the information at this end of the spectrum is highly qualitative, open to interpretation by various entities and includes health diary entries, journal entries, activity logs, diet logs, messages received and sent, and the like. Along the data spectrum are data types such as “feel good” measurements, surveys (e.g., SF-36 and other quality of life surveys), specific data on diet and exercise (e.g., “I had a steak today and walked three miles”), information relating to a patient's mental health, pain management information, and so on. Some of these data, such as the quality of life surveys may appear “subjective” but are actually well validated and reproducible, and can be classified as interpretable/actionable data. In the described embodiment, the data entered by a patient or by the patient's healthcare team, regardless of where the data fall along the spectrum are entered into patient's personal health record 202 in a timely manner. These data are not stored or buffered for any significant amount of time before they are entered into the patient's health record. Effectively, the health data of a patient are updated live and close to real time.

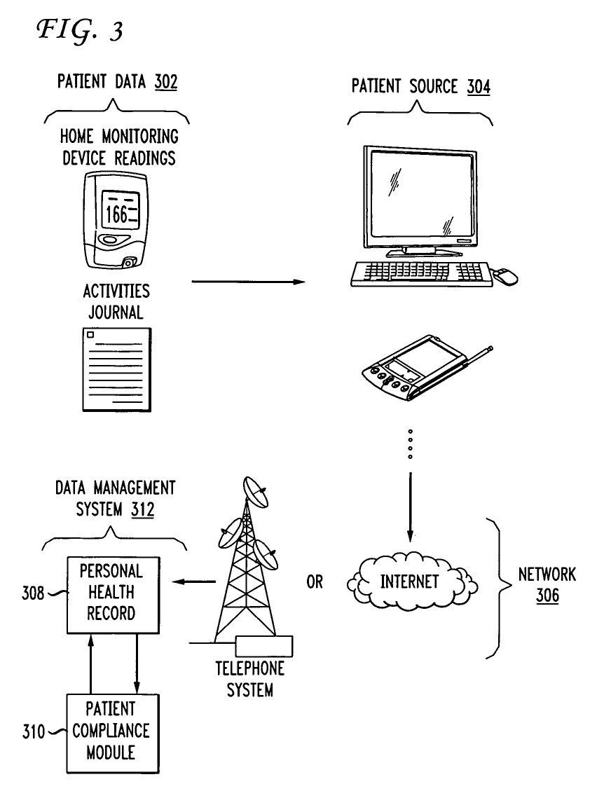

FIG. 3 is a block diagram showing software logic components for a patient compliance system in accordance with one embodiment of the present invention. Patient data 302 are entered at a patient source 304, such as a computer or biometric device. Data are sent over a network 306 and entered into a personal health record 308. A patient compliance module 310 examines and tracks data 302. Components 308 and 310 are at a health data management system 312. These data can also be examined by a caregiver (not shown). Compliance module 310 contains logic that calculates whether data 302 entered meets the criteria of a treatment plan or other health/medical instructions in personal health record 308. If appropriate, an automated response is sent to the patient. In a preferred embodiment, an automated response results from processing data that fall closer to the well-validated data end of the data spectrum described above, such as biometric readings. In a preferred embodiment of the present invention, a doctor or other professional healthcare provider provides feedback and responses to the patient regarding compliance based on data 302 entered by the patient. In another preferred embodiment, all intervention and feedback is “signed off” by a healthcare professional before being sent to the patient.

In another preferred embodiment, a hybrid of automated analysis of data by the system and intervention by a doctor to contact patients that are in greatest need of guidance and instruction with respect to compliance is implemented. There will always be patients who are “furthest out” and at greatest risk of bouncing back into the healthcare system and requiring further expensive healthcare. The patient compliance monitoring system of the present invention identifies those patients, for example, by finding the largest discrepancies between treatment plan parameters and actual parameters drawn from live data. Another way of identifying those patients is by looking at the history of messages sent to the patient. Regardless of the method, the system informs the doctor that more interactive or hands-on intervention may be needed. This important feature helps prevent the worst-case patients from getting worse and is intended to keep them from needing further expensive healthcare. In all monitoring methods and combinations, the monitoring is continuous unless explicitly terminated by an authorized party.

Another feature of the patient compliance system of the present invention is its reliance on a rich set of data and statistics for each patient. The more live data, historical data, and statistics there are, the more effective the patient compliance monitoring system is. In a preferred embodiment, these data and information are stored in or accessible via a patient health record. From these data, the healthcare data management system derives a wide range of statistical data on each patient. The range and depth of the statistical data can vary from system to system and from patient to patient. However, in a preferred embodiment, patient compliance module 310 determines compliance using a combination of data and statistics on each patient. At a basic level, it is necessary that some type of data are sent by the patient and that it is being done on a regular basis. From these data, mean, median, standard deviations, and other analysis can be done.

In a preferred embodiment of the present invention, a patient is notified of compliance status by receiving feedback from the data the patient or others have entered into the knowledge database. The feedback is typically in the form of a message. Another form of feedback is a reward, discussed in further detail below. In the present invention, feedback to a patient is customized for the patient, is more balanced, and is timely. Feedback is customized because the patient's personal health record, statistics, and previous feedback are all considered in the calculation in light of the patient's treatment program, as embodied in the logic contained in the patient compliance module and incorporated into the personal health record. The set of instructions and parameters is provided by a doctor or professional caregiver, and consists of prescriptions, healthcare regimens, etc. For example, for a patient with a heart condition, a treatment plan would likely include blood pressure parameters, or for a diabetic patient, a plan would include parameters on blood sugar. Messages to the patient are based on data and statistics that are germane only to the patient.

Feedback to a patient is balanced. It can include messages that are cautionary, assertive, and provide an “alert” if a patient is falling behind in a treatment program. The feedback can also provide further guidance on how to improve compliance or alternative practices that may be more suitable to the patient and help her stay with the curative scheme. Generally, feedback is positive, supportive and encouraging in tone, if the patient's data and statistics indicate he is on track, within a “goal range” (attaining and staying within the goals) or going beyond expectations with respect to a treatment program in which case messages can be congratulatory. Messages can also be informational or educational and be used to simply let the patient know that someone is being attentive to the patient's needs and to the data on activities, daily routine, and so on, that the patient is taking the time and effort to enter (e.g., “This month is Diabetes Month—check with your clinic for special programs they may be holding.”). Such informational/educational messages provide occasional encouragement can be therapeutic to the patient. These types of messages can also provide additional or modified instructions and guidelines regarding the treatment program. The overall effect is that that feedback to the patient is balanced, rather than negative or disciplinary in nature, as is the case in many present compliance systems and methodologies.

Finally, the patient receives feedback frequently and soon after he or she has entered data. In the era of instant messaging, mobile devices for e-mail, and increasingly pervasive wireless Internet connections, receiving feedback on compliance in a few hours or sooner provides significant motivation to patients to enter data and see how they are doing with their treatment program. It reinforces one of the underlying strategies of the present invention: making patients want to be more compliant with their treatment programs instead of forcing compliance on them.

In another embodiment of the present invention, the patient is not the only one being watched. The monitoring system is hierarchical. In a typical scenario the patient is at the bottom of the hierarchy, preceded by a doctor, who may be part of a doctor group, preceded in the hierarchy by one or more hospitals, which are monitored or observed by insurance companies. A hypothetical and simplified scenario of the hierarchical monitoring schema enabled by the patient compliance system of the present invention involves a patient, her doctor, and a doctors group to which the doctor belongs, and a hospital. A doctor group is given a score for its overall performance by a hospital. The score is determined by the performance level of each doctor in the group. The performance level of an individual doctor is measured, in this simplified illustration, by the content and type of messages sent to the doctor's patients. If a doctor has patients who are, for whatever reason, very compliant to their treatment programs as indicated by a high number of congratulatory messages, that doctor and his team of caregivers are deemed to be doing a good job. This specific metric can be used as one indicator (among several) to gauge the performance of each doctor in the group. Similarly, if a doctor within a group is having difficulty getting patients to comply with their treatment programs, this is similarly indicated by the number and quality of the messages sent to the patients. In this manner, doctor groups or individual doctors can be rewarded or recognized for having a ‘high patient treatment compliance’ rate. All feedback, including messages to a patient, and messages from a patient, are stored in or accessible via the knowledge database and associated with the patient's personal health record. In a preferred embodiment, all feedback is stored in the health record. These messages are utilized to measure the effectiveness of treatment programs and, to some extent, the quality and effectiveness of services provided by healthcare professionals.

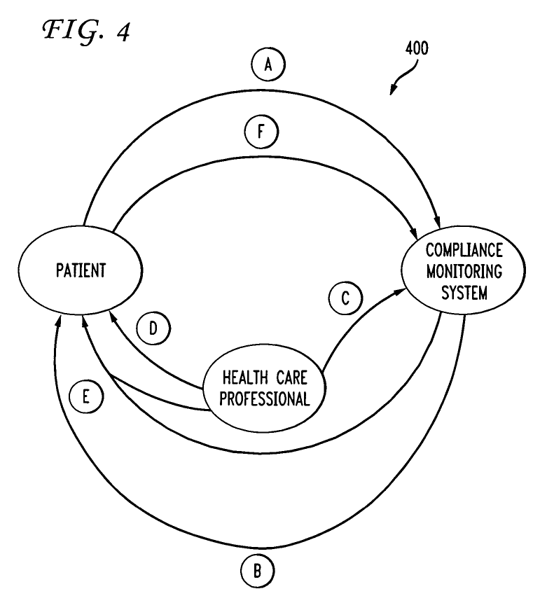

FIG. 4 is a diagram of a patient compliance monitoring feedback loop of the present invention. Monitoring compliance system 402 of the present invention is interactive and implements a feedback loop 400 in which a patient 404 enters a response or feedback to the messages he or she receives. As described below, this feedback is another type of data that the patient can enter into the personal health record.

Feedback loop 400 starts with step A where patient 404 enters data or information into compliance system 402. At step B, compliance system 402 sends a compliance monitoring message to patient 404. A healthcare professional 406 can view or be notified of the data entered by patient 402 as shown at step C. Healthcare professional 406 can then send a monitoring message to patient 404 at step D. Patient 404 can also receive a message that is partly automated but has some degree of intervention by healthcare professional 406 as shown at step E. Thus, patient 404 receives customized, timely, and balanced messages regarding the patient's compliance with a treatment program. Patient 404 can respond to compliance monitoring system 402 using a PC or other IP-enabled device as shown at step F, thereby completing the interactive feedback loop of the present invention.

A typical response can vary widely, it can be a comment on the message that the patient received, the patient's opinion on whether she thinks she can follow the recommendation or suggestion in the message, checking a box indicating that the patient has complied with the recommendation or order, and so on. The messages or responses from the patient are also stored in the system and can be used to measure the effectiveness of a treatment program or of a doctor's performance and essentially another type of data that can be used to measure compliance. As described above, the present invention depends on the quantity and richness of the live data entered by the patient, the depth and range of historical data, and the statistical data that can be derived from these data.

One of the underlying strategies of the present invention is making patients want to be compliant with their treatment programs. One method of supporting this is rewarding the patient for 1) entering data relevant to the patient's treatment program and 2) for actually complying with a treatment program. As mentioned above, a patient with a chronic condition is supposed to follow health/medical instructions from a professional. Those who strictly adhere to a treatment program are less likely to require further expensive medical attention. These patients save money for insurance companies, government agencies, employers, and themselves, and they lessen the financial strain on the healthcare industry in general. Data relating to a patient's compliance to a treatment program, collectively by the present invention can be used by these entities to reward superior compliance. A patient can also enter information describing educational activities he or she has done, such as going to classes or support groups, that are beneficial to or supplement a treatment program. More generally, a patient can enter data describing good behavior by the patient (perhaps accompanied by proof or verification) for which the patient will be rewarded.

Similar to a process in which messages and feedback are composed and sent to a patient based on his or her compliance, a score or other compliance indicator can be derived for each patient. This score or indicator can be used in a variety of ways by entities in the healthcare industry to reward the patient. Similarly, the score can be used in a punitive or disciplinary manner for those who are non-compliant. In another embodiment, the number of messages and qualitative nature of those message can be examined to determine whether a patient should be rewarded. This is possible because all the messages are saved in the knowledge database or related storage area. In a simplified illustration, a message to a patient can be categorized as positive, negative, or neutral. If a patient receives only positive and neutral messages or a minimum number of negative messages over a pre-defined time period, the patient may be entitled to a reward. The type of reward can vary widely and depend on the entity or party providing it. The objective in all cases however would be to reinforce and encourage compliance with a treatment program.

In the patient compliance system of the present invention, it is intended that the patient know or be aware that he or she is being observed and that their compliance with a treatment program is being measured each time they make an update to their personal health record. This awareness by the patient typically has a beneficial effect on patient compliance behavior. This is particularly true if the patient receives timely feedback (whether automated from a professional provider, or a combination of both), for example, on the same day the patient data were entered.

Although certain aspects of the patient compliance system of the present invention are automated, namely examining the patient data, calculating statistics, and comparing to a treatment program, the system is not intended to replace the judgment of a doctor or the participation of professional caregivers. Doctors and other professional caregivers can modify the treatment program of a patient based on the patients biometric readings and other data in the personal health record. Their analysis and intervention is critical to the overall effectiveness of the present invention.

Although the foregoing invention has been described in some detail for purposes of clarity of understanding, it will be apparent that certain changes and modifications may be practiced within the scope of the appended claims. Furthermore, it should be noted that there are alternative ways of implementing both the methods and systems of the present invention. For example, data can be entered by the patient and need not include biometric data but rather only narrative, journal/diary type data. Data can also come from a healthcare professional. The parameters of the treatment plan can be inherent in the patient's personal health record and need not be an explicit set of instructions. The patient compliance module can operate on a separate system from the health data management system but share the personal health record with the management system. Accordingly, the present embodiments are to be considered as illustrative and not restrictive, and the invention is not to be limited to the details given herein, but may be modified within the scope and equivalents of the appended claims.

We claim:

1. A method of encouraging a patient to follow a treatment plan, the method comprising:
receiving health-related data from the patient at a compliance monitoring system;
analyzing the data to determine whether the patient is compliant with the treatment plan stored at the compliance monitoring system;
sending content to the patient wherein the content is derived from whether the patient is compliant with the treatment program; and
enabling the patient to respond to the content by sending a feedback message to the compliance monitoring system.
